(19) United States
(12) Patent Application Publication
(10) Pub. No.: US 2009/0299379 A1
(43) Pub. Date: Dec. 3, 2009

(54) MYRINGOTOMY INSTRUMENT

(76) Inventors: Yeshayahu Katz, Haifa (IL); Roni Shabat, Kibutz Israel (IL)

Correspondence Address: YORAM TSIVION, P.O. BOX 1307, PARDES HANNA, 37111, IL

(21) Appl. No.: 12/127,834

(22) Filed: May 28, 2008

(51) Int. Cl. A61F 11/00

(52) U.S. Cl. 606/109

(57) ABSTRACT

An instrument for performing myringotomy operations consists of a device for incising, suctioning and grasping a tympanostomy tube (ISGTT) attached to a gripping handle. The ISGTT consists of a gripper and a pushing and suctioning member (PS) surrounded by an external tube. Peripheral fingers of the gripper press against the surface of the tubular segment of the tympanostomy tube for grasping. An incising blade disposed within the lumen of the PS is movable coaxially with the bore of the tympanostomy tube. Blade positioning selector located at the proximal end of the ISGTT provides for drawing and withdrawing the incising blade through the bore of the gripped tympanostomy tube. A lever pivotally attached to the griping handle is used to control grasping and releasing of the tympanostomy tube when properly placed.

### FIELD OF THE INVENTION

The present invention relates in general to myringotomy. More particularly, the present invention relates to an instrument providing for incising the eardrum, suctioning the fluid released from the middle ear passage and positioning the tympanostomy tube in place.

### BACKGROUND OF THE INVENTION

Treating middle ear infection, or otitis media occasionally involves a surgical operation in which an incision is made through the tympanic membrane. Any fluid and/or usually thickened secretions are removed. A tympanostomy tube is typically inserted into the incision to keep the middle ear passage aerated and permit drainage of fluid to the outer ear meatus. Normally such an operation requires a number of devices to be employed by a surgeon who, as a result, occasionally must redirect his/her line of vision from the patient's ear. An integral instrument providing for a significant number of activities typically associated with such an operation may aid the surgeon to remain focused in the patient's ear during the operation process.

U.S. Pat. No. 3,913,584 discloses an instrument providing for most of the activities typically performed during myringotomy and the insertion of tympanostomy tube into the eardrum. The disclosed device consists of an outer sleeve surrounding an inner tube. The inner tube, the distal end tip of which is sharpened, is adapted for releasably holding the tympanostomy tube across its distal end, such that its sharpened end tip protrudes from the tympanostomy tube outwards. The inner tube also provides for suctioning the fluid released from the middle ear passage following the incision made in the tympanic membrane by means of its sharpened tip. The tympanostomy tube is released from the inner tube and placed through the incision by means of the moveable sleeve. However, the disclosed device does not provide the surgeon with the required control for correctly positioning the tympanostomy tube through the incision in the eardrum. Furthermore its sharpened tip may harm the patient's ear and/or the surface of the tympanostomy tube. Therefore, a more user-friendly instrument providing for orienting and positioning of the tympanostomy tube in the eardrum that is less hazardous to the ear passages and or the tympanostomy tubes is called for.

### BRIEF DESCRIPTION OF THE DRAWINGS

FIG. 1 is an isometric view of a myringotomy instrument according to a preferred embodiment of the present invention;

FIG. 2 is an isometric view of a segment of the device for incising, suctioning and tympanostomy tube holding of the myringotomy instrument shown in FIG. 1;

FIG. 3 is a sectional view of the segment of the device for incising, suctioning and tympanostomy tube holding shown in FIG. 2;

FIG. 4 is a front side view of the distal end of the myringotomy instrument shown in FIG. 1;

FIG. 5 is an isometric view of the proximal segment of the device for incising, suctioning and tympanostomy tube holding of the myringotomy instrument shown in FIG. 1;

FIG. 6 is a sectional view of a segment of the myringotomy instrument shown in FIG. 1;

FIG. 7 is a schematic presentation of a tympanosomy tube on which an auxiliary spacer is placed;

### DETAILED DESCRIPTION OF THE PRESENT INVENTION

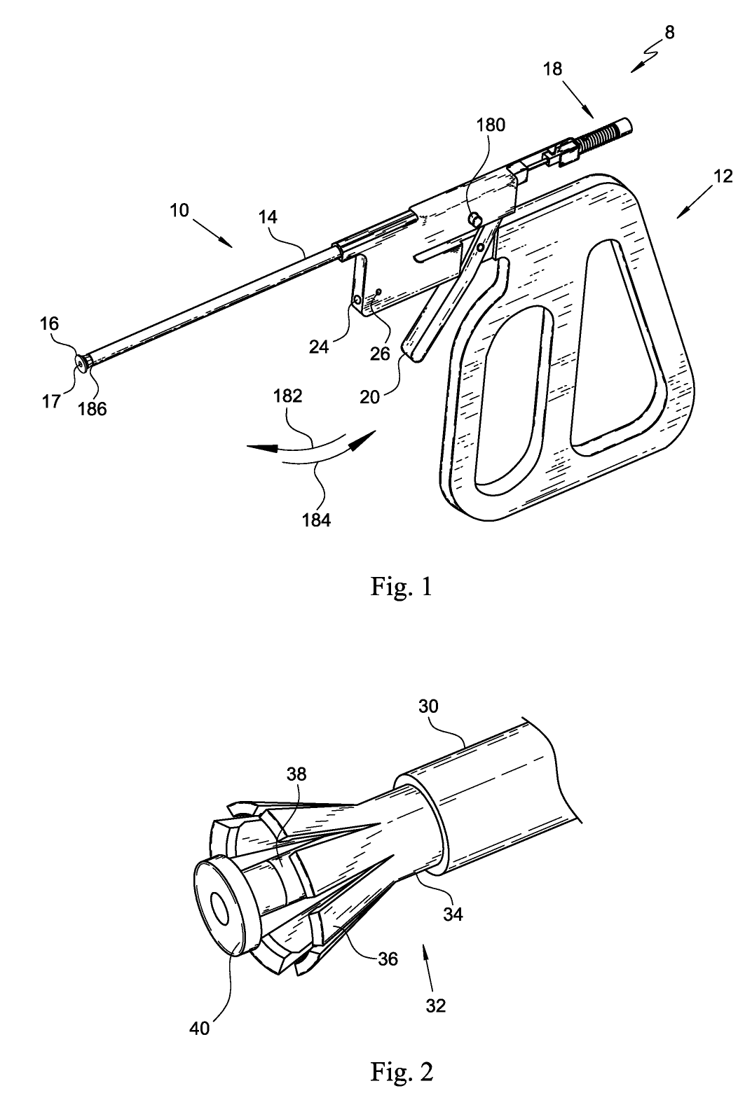

In accordance with the present invention an instrument for performing myringotomy operations is provided. The instrument of the invention provides for incising the tympanic membrane, suctioning the fluids released from the middle ear passage and positioning the tympanostomy tube (TT). An instrument provided in accordance with a preferred embodiment of the present invention is hereby described with reference to FIGS. 1-3. All the directional notations such as upwards or downwards referred to hereinafter relate only to the scenario indicated in the referenced drawings, as the devices in reality can be arbitrarily oriented in any direction. In FIG. 1 an isometric view of instrument for performing myringotomy 8 consisting of a device for incising, suctioning and grasping a TT (ISGTT) 10 attached to gripping handle 12 is shown. ISGTT 10 has external tube 14 in which the TT holder and the incising blade, both not shown, are installed. Gripped TT 16 protrudes from the distal end of ISGTT 10. The length of external tube 14 is such that the distance between the distal end of ISGTT 10 and distal surface of gripping handle 12, referred to hereinafter as the clearance length, exceeds a predefined value. Typically the clearance length is not smaller than 8 centimetres. Therefore, the clearance between the patient's ear and gripping handle 12 provides the surgeon with conveniently rotating and translating ISGTT 10 within the external ear passage without blocking the line of sight. The incising blade is drawn outwards and is withdrawn through bore 17, by means of blade positioning selector 18 located at the proximal end of ISGTT 10. Lever 20 that is pivotally connected to the upper side of gripping handle 12 provides for grasping and releasing TT 16 and for pushing TT 16 through the incision made in the tympanic membrane, as is further described infra. Negative pressure for suctioning is provided through aperture 24 by means of a vacuum tube of prior art, not shown, when slip fitted into aperture 24. Fluids and air passageway, not shown, connects between aperture 24 and the lumen of the pushing and suctioning member (PS) of the TT holder, not shown. The lumen of the PS has a fluid connection with bore 17, as is further described infra. The sealing of working aperture 26, which is an additional aperture of this passageway, by a user's finger, effects negative pressure at the distal end of bore 17.

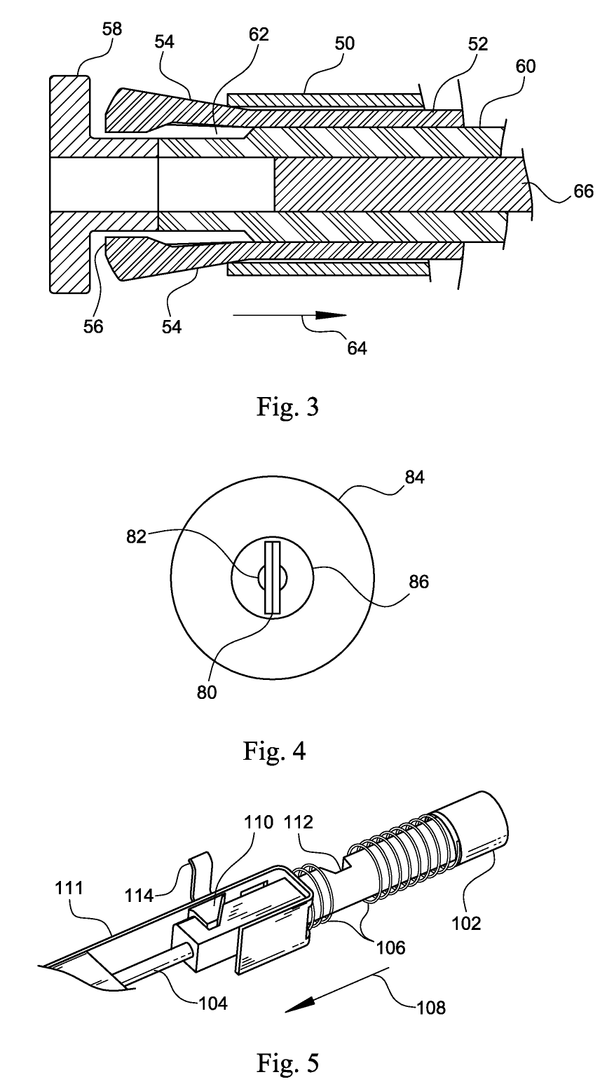

In FIG. 2 an isometric view of a segment of the ISGTT shown in FIG. 1 is shown. Out of the distal end of external tube 30 TT holder 32 is forwardly protruding. A TT holder of the invention typically consists of a gripper, a PS and a base. A gripper according to the invention has an engaging surface surrounding the tubular segment a TT for its grasping. The gripper in accordance with a preferred embodiment of the present invention consists of tube 34 whose distal end is furcated into a circle of inwardly bendable fingers 36. PS 38 is another tube coaxially disposed within the lumen of tube 34. TT 40 is appended to PS 38 such that its bore is coaxial with PS 38. In FIG. 3 a sectional view of the segment of the ISGTT described above is shown. External tube 50 surrounds tube 52 of the gripper, while the gripper is translatable within tube 52. Circle of fingers 54 the outside surface of which is conical, protrudes out of the distal end of tube 50. Inwardly protruding teeth 56 of fingers 54 provides for clutching TT 58 attached to the distal end of PS 60 and for sealing the connection between TT 58 and PS 60. The inner surfaces of teeth 56 constitute a divided engaging surface of the gripper. The external diameter of PS 60 conforms with the inner diameter of tube 52 except for its extreme end the diameter of which equals the diameter of the tubular segment of TT 58. Clearance 62 provides for inwardly bending fingers 54 and for forcing respective surfaces of teeth 56 against the surface of TT 58, when moved backwards in the direction of arrow 64 into a gripping position. Alternatively, a gripper consists of one or more rings the diameter of which is changeable, or gripping fingers translatable in directions normal to the axis of the TT. Incising blade 66 is disposed in the lumen of PS 60. The blade is connected by means of a shaft, not shown, to blade positioning selector 18 shown in FIG. 1 to which reference is again made.

Reference is now made to FIGS. 4-6 in which further details of the ISGTT shown in FIG. 1 are provided. In FIG. 4 a front side view of the ISGTT is shown. Blade 80 that is attached to shaft 82 is either drawn out of bore 86, or withdrawn into the lumen of the PS, not shown. Bore 86 is centered at the distal face of TT 84. Typically, the diameter of the external tube surrounding the gripper and the maximal diameter of the cone formed by the outermost surfaces of the gripper fingers when fully retracted do not exceed the diameter of the distal surface of TT 84. Namely, the distal segment of the ISGTT ranging from its distal end up to the surface of the gripping handle is confined by a cylindrical projection of TT 84 coaxial with bore 86. Therefore, the external surface of TT 84 is the front end of an ISGTT of the invention facing any part of an ear of a patient in the course of the surgical process, whenever the incising blade is withdrawn. Furthermore, the external tube and/or the ISGTT may touch the oval speculum typically employed for maintaining sterility and protecting the ear only by one of its points.

In FIG. 5 blade positioning selector 100 is shown. Lever 102 is attached to shaft 104 that is further connected on its other end to the incising blade, not shown. Biasing spring 106 provides for retracting lever 102 and therefore withdrawing the incising blade back into the lumen of the PS, not shown. Pushing lever 102 in the direction of arrow 108 draws the incising blade. Stopper 110 resiliently attached to frame 111 is released when forced by lever 102 in this direction. Lever 102 is continuously moved further up to the point that stopper 100 faces recess 112. It is sprung into recess 112 whereby the movement of lever 102 is stopped. At this position, which is the drawn position, the incising blade is drawn out to its full extent. Releasing stopper 110 by means of lever 114 causes lever 102 to retract to its initial position and concomitantly withdraw the incising blade. In a drawn position the length of the segment of the incision blade protruding out of the TT is not smaller than a predefined drawing length. Thus the TT and/or the ISGTT do not block the line of sight of the surgeon who may conveniently incise the tympanic membrane. A drawing length of two centimetres normally conforms to the small diameters of the above-mentioned speculums.

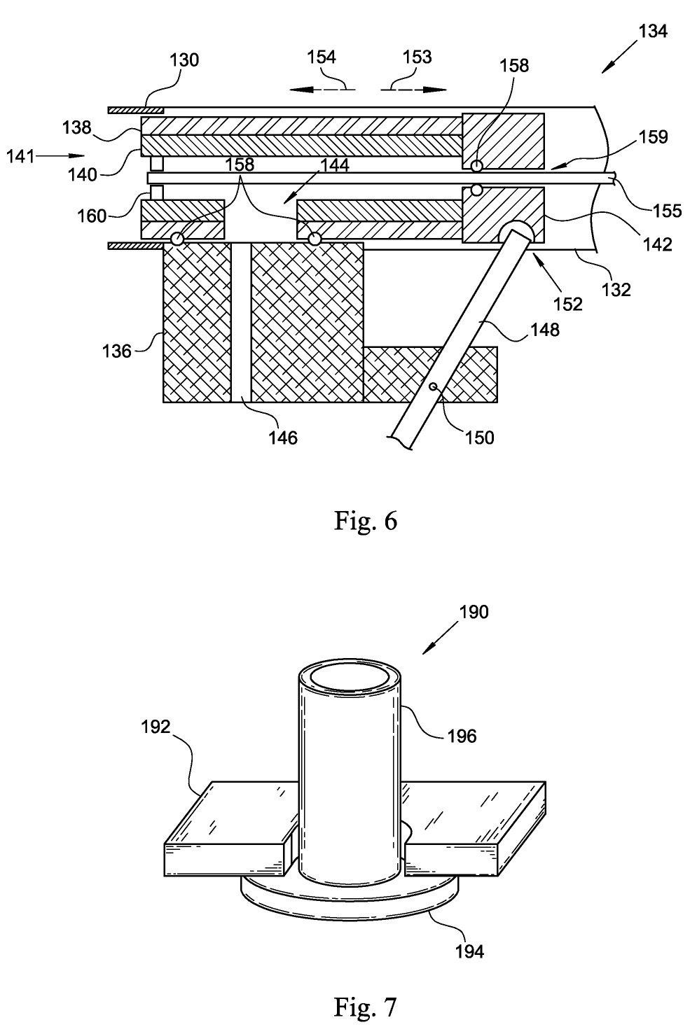

In FIG. 6 a sectional view of a segment of the instrument for performing myringotomy shown in FIG. 1 is shown. External tube 130 and frame 132 of ISGTT 134 are attached to gripping handle 136. Gripper 138 and PS 140 are attached to TT holder base 142. Optionally gripper 138 and PS are also attached to each other. TT holder 141 is movably disposed in the lumen of external tube 130. The lumen of PS 140 is hermetically sealed except for the aperture, not shown, in its distal end and the longitudinal aperture 144 leading to the fluid and air passageway 146. Lever 148 is connected to gripping handle 136 by hinge 150. One end of lever 148 is engaged in recess 152 located at the bottom of TT holder base 142, whereas its other end is downwardly protruding. Therefore, translational motion of TT holder 141 in the directions of arrows 153 or 154 is imparted by respective pivotal rotations of lever 148. Shaft 155 connecting the incising blade and the blade positioning selector is movably disposed in the lumen of PS 140. Optionally o-rings 158 hermetically seal apertures 156 and the connection between gripping handle 136 and PS 140 respectively. One or more spacers such as spacer 160 are optionally disposed in the lumen of PS 140 for keeping shaft 154 coaxial with PS 140. The gripper and the PS can be moved independently according to the invention, such as by employing separate bases instead of common TT base holder 142, an engagement mechanism and one or two pivotal and or translational dedicated levers, as known in the art.

For operating an instrument for performing myringotomy of the invention first a TT must be inserted and gripped by the distal end of the ISGTT. For this purpose a TT is vertically placed having its distal face on a horizontal surface. The gripper is opened by pulling lever 148 to its maximal extent in the direction of arrow 153. Then the tubular segment of the TT is inserted into the clearance surrounded by the fingers of the gripper such that the distal face of the PS engages the TT.

Optionally, an auxiliary spacer is employed, as can be seen in FIG. 7 to which reference is now made. TT 190 is vertically posted having spacer 192 placed over flange 194. The width of spacer 192 conforms to the difference between the length of tubular segment 196 and the distance between the distal end of the ISGTT and the distal face of the PS. Reference is again made to FIG. 6. The distal end of the ISGTT is pressed against the spacer while lever 148 is concomitantly pushed to its maximal range in the direction of arrow 154. At this stage in which the TT is gripped and the gripper and the TT holder are in a retracted position the instrument for performing myringotomy is ready to be inserted into an external ear passage of the treated patient.

Reference is again made to FIG. 1. Range selector 180 provides for limiting the pivotal rotations of lever 20 such that three distinct positions of the gripper are provided. When range selector 180 is pulled outwards as is shown in FIG. 1 lever 20 can be rotated up to its maximal extents in the directions of arrows 182 and or 184 respectively. For gripping a TT, lever 20 is maximally rotated in the direction of arrow 184 by which the gripper moves to an open position. Then lever 20 is maximally rotated in the opposite direction to bring the gripper into a retracted position, as is described hereinabove. At a retracted position selector 180 is pushed inwards which results a limited rotational angle in the direction of arrow 182. Rotating lever 20 by such an angle causes TT 16 to be pushed away from aperture 186 by a distance conformal to the length of the tubular segment of TT 16. At this stage in which the gripper is in an intermediate gripping position TT 16 is still being held by the gripper providing for improving the positioning of TT 16 through the incision made in the eardrum. Two stoppers, not shown, limit the translational range of the TT holder in both directions, which in turn limit the angles of respective pivotal rotations of lever 20.

The ISGTT of the invention is normally made of materials such as stainless steel or plastic resins typically employed for manufacturing disposable medical devices. The gripping handle is typically made of such plastic resins. Mounting of a ISGTT onto a gripping handle need not be limited to the manufacturing phase of an instrument for performing myringotomy of the invention but can be also carried out by a user prior to its usage in the operating room.

1. A device for incising a tympanum, suctioning secretions form the ear and grasping a tympanostomy tube, wherein said tympanostomy tube (TT) has an external surface, a tubular segment and a bore, said device comprising
a gripper having an engaging surface for gripping said TT;
a pushing and suctioning member (PS) having a lumen and an aperture having a circumferential surface onto which said gripped TT is appended, wherein said bore of said TT forms a continuum with said lumen of said PS;
an incision blade stored within the lumen of said PS, wherein said incision blade is movable coaxially within said bore, and
wherein said engaging surface is pressed against the external surface of said TT for gripping said TT.

2. A device as in claim 1, and wherein the diameter of the distal end of which is not larger than the diameter said gripped TT, and wherein the length of said segment is not smaller than a predefined clearance length.

3. A device such as in claim 1, wherein a fluid and air passageway is connected to said PS, and wherein the lumen of said PS and the lumen of said passageway form a continuum, and wherein said passageway further comprising at least one aperture adapted for connecting to a source of negative pressure.

4. A device as in claim 1, further comprising a blade positioning selector connected to said incision blade whereby said incision blade is drawn from said PS.

5. A device as in claim 1, and wherein said gripper is translatable coaxially with said bore of said TT between at least two positions, and wherein the distance between said at least two positions is not smaller than the length of said tubular segment.

6. A device as in claim 5, and wherein a lever pivotally attached to a gripping handle is engaged to said gripper for translating said gripper.

7. A device as in claim 5, and wherein said PS is translatable concomitantly with said gripper.

8. A device as in claim 1, wherein at least one segment of said engaging surface is movable in a direction substantially normal to the axis of said bore.

9. A device as in claim 1, wherein said PS is translatable independently from said gripper.

10. A device as in claim 1, wherein said incision blade is movable into a drawn position whereby a segment of said incision blade protrudes from said bore to a predefined length.
